(19) United States
(12) Patent Application Publication
(10) Pub. No.: US 2008/0139844 A1
(43) Pub. Date: Jun. 12, 2008

(54) Method Of Preparing Acrylic Acid From Propane In The Absence

(76) Inventors: Jean-Luc Dubois, Millery (FR); Dominique Garrait, Charly (FR); Anne Le Gall, Francheville (FR); Guy Bazin, Lyon (FR); Stephanie Serreau, Oullins (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/813,148

(22) PCT Filed: Dec. 16, 2005
(86) PCT No.: PCT/FR05/03168

§ 371 Date: Jun. 29, 2007

(30) Foreign Application Priority Data
Dec. 30, 2004 (FR) 0414082

(51) Int. Cl. C07C 51/16

(52) U.S. Cl. 562/549

(57) ABSTRACT

The invention relates to a method of preparing acrylic acid by selective oxidation of propane in a circulating fluidised bed or in a fluidised bed in the presence of a catalyst having structure Mo₁VₐX_{b}Z_{c}Si_{d}Oₓ(I), wherein X represents tellurium or antimony and Z represents niobium or tantalum, and in which: —a is between 0.006 and 1, inclusive; —b is between 0.006 and 1, inclusive; —c is between 0.006 and 1, inclusive; d is between 0 and 3.5, inclusive; and x is the amount of oxygen bound to the other elements and depends on the oxidation states thereof, which is performed under partial propane conversion conditions and without the introduction of water vapour into the initial gas mixture used to supply the reaction.

The present invention relates to a process for preparing acrylic acid by selective oxidation of propane, without introduction of steam into the reaction gases.

Patent application EP 608 838 describes the preparation of an unsaturated carboxylic acid from an alkane by a vapor-phase catalytic oxidation reaction in a cofed fixed bed reactor in the presence of a catalyst comprising a mixed metal oxide, the essential components of which are: Mo, V, Te, O and at least one element chosen from the group consisting of Nb, Ta, W, Ti, Al, Zr, Cr, Mn, Fe, Ru, Co, Rh, Ni, Pd, Pt, Sb, Bi, Bo, In and Ce, these elements being present in precise proportions. This patent application indicates that the presence of a significant amount of steam in the reaction mixture is advantageous from the viewpoint of the conversion and of the selectivity for acrylic acid. However, no information is given with regard to the amount of byproducts formed during the reaction, whereas this aspect is a crucial point in the industrial preparation of acrylic acid.

Japanese patent application 2000-256257 describes the conversion of propane to acrylic acid in redox mode over an MoVSbNb catalyst. It is clearly indicated that the presence of steam is preferable in order to have a better yield of acrylic acid. Thus, a water/propane molar ratio greater than 0.5 is desirable.

A description was given, in patent application US 2004/0138500, of a process for the partial oxidation of propane to acrylic acid in the presence of a multimetal oxide catalyst using a starting gas mixture composed of propane, molecular oxygen and at least one diluent gas which comprises steam.

A description was given, in European application EP 1238 960, of a process for the preparation of acrylic acid from propane, in which a gas mixture deprived of molecular oxygen and comprising propane, steam and optionally an inert gas is passed over a solid composition with the structure: Mo₁VₐTe_{b}Nb_{c}Si_{d}Oₓ in order to oxidize the propane according to the redox reaction:

SOLID_{oxidized}+PROPANE→SOLID_{reduced}+ACRYLIC ACID

A description was given, in international application WO 04/024666, of a process for the production of acrylic acid in which a gas mixture comprising propane, molecular oxygen, steam and, if appropriate, an inert gas is passed over a tellurium-based catalyst of structure: Mo₁VₐTe_{b}Nb_{c}Si_{d}Oₓ and in which the propane/molecular oxygen molar ratio in the starting gas mixture is greater than or equal to 05.

A description was given, in international application WO 04/024665, of the preparation of acrylic acid in which a gas mixture comprising propane, steam, optionally an inert gas and/or molecular oxygen is passed over an antimony-based catalyst of structure: Mo₁VₐSb_{b}Nb_{c}Si_{d}Oₓ in order to oxidize the propane to acrylic acid, and, when molecular oxygen is introduced, the propane/molecular oxygen molar ratio in the starting gas mixture is greater than or equal to 0.5

However, in the processes of the prior art, it was essential to introduce steam into the gas mixture brought into the presence of the catalyst. This is because the state of the art clearly indicates that the presence of water is necessary for satisfactory operation of the catalyst and in order to achieve good selectivities.

It has now been found, and it is this which forms the subject matter of the present invention, that the selective oxidation of propane to give acrylic acid, in a circulating fluidized bed or in a fluidized bed, in the presence of a metal oxide catalyst, under conditions comprising a low degree of conversion of the propane and under conditions making it possible to dispense with the introduction of steam, results in a substantial improvement in the process.

This is because the process is found to be greatly improved in terms of economy in the amount of energy destined for the evaporation of the water and then subsequently in the amount of energy destined for its removal from the reaction products.

Furthermore, the presence of water facilitates the sublimation of some constituents of the catalyst and also causes pulverulent catalysts to tend to agglomerate, followed by setting solid, which results in the operation of the reaction being disrupted. These phenomena are thus reduced.

The process is also found to be improved owing to the fact that the acrylic acid is more easily separated from an effluent when the latter is as concentrated as possible, this effluent comprising, apart from the acrylic acid, unconverted reactants, steam produced by the reaction and also all the reaction byproducts, in particular byproducts whose formation is promoted by the presence of water (such as, in particular, propionic acid or acetone, which are formed by hydration of the intermediate propylene of the reaction). Thus, the process is found to be improved by a reduced formation of certain reaction byproducts.

Thus, the present invention consists of the selective oxidation of propane to give acrylic acid, in a circulating fluidized bed or in a fluidized bed, in the presence of a catalyst with the structure,

Mo₁VₐX_{b}Z_{c}Si_{d}Oₓ (I)

in which X is tellurium or antimony and Z is niobium or tantalum, and in which;
a is between 0.006 and 1, limits included;
b is between 0.006 and 1, limits included;
c is between 0.006 and 1, limits included;
d is between 0 and 3.5, limits included; and

x is the amount of oxygen bonded to the other elements and depends on their oxidation states,

under conditions for the partial conversion of the propane and without introduction of steam into the initial gas mixture feeding the reaction.

Thus, the aim of the invention is to provide a process for the production of acrylic acid from propane, in the presence of molecular oxygen, which makes it possible to obtain good selectivity for acrylic acid while limiting the formation of undesirable reaction byproducts, such as propionic acid and acetone.

It has been found that this aim can be achieved by passing a gas mixture comprising propane, oxygen and, if appropriate, an inert gas over a specific catalyst. In particular, when the operation is carried out in a circulating fluidized bed, the operation is carried out under conditions such that the oxygen of the gas mixture is in a substoichiometric proportion with respect to the propane, which allows the catalyst to act as a redox system and to supply the missing oxygen in order for the reaction to be carried out satisfactorily.

The conversion of the propane to acrylic acid by means of the catalyst is carried out by oxidation, probably according to simultaneous reactions (1) and (2): conventional catalytic reaction (1):

CH₃—CH₂—CH₃+2O₂→CH₂═CH_COOH+2H₂ (1)

redox reaction f:

SOLID_{oxidized}+CH₃—CH₂—CH₃-→SOLID_{reduced}+CH₂═CH—COOH (2)

The proportion of inert gas introduced, which can, for example, be nitrogen or else carbon dioxide, is not critical and can vary within wide limits. Other gases, such as unconverted propane, propylene or light hydrocarbons, can be present in the gas mixture feeding the reaction.

Generally, reactions (1) and (2) are carried out at a temperature of 200 to 500° C., preferably of 250 to 450° C., more preferably still of 350 to 400° C.

It is advantageous to operate at a partial degree of conversion of the propane, in order to limit the formation of the reaction byproducts.

The pressure in the reactor is generally from 1.01×10⁴ to 1.01×10⁶ Pa (0.1 to 10 atmospheres), preferably from 5.05×10⁴ to 5.05×10⁵ Pa (0.5-5 atmospheres).

The residence time in the reactor is generally from 0.01 to 90 seconds, preferably from 0.1 to 30 seconds.

According to a specific embodiment of the invention, the reactor used can be a circulating bed reactor as described previously in international application WO 99/03809, in which the reaction region is composed of 2 parts: a fluidized bed and a riser, and the regeneration region, which comprises a fluidized bed.

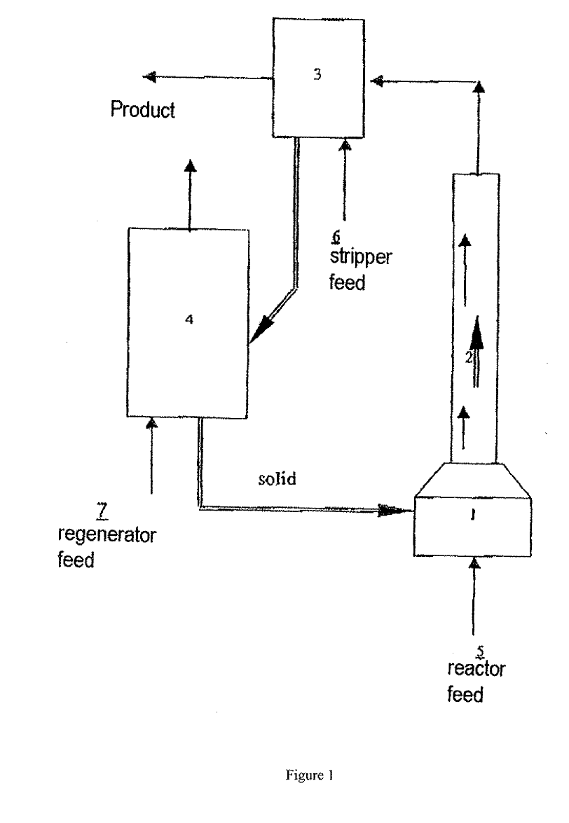

More particularly, use is made of a circulating fluidized bed reactor [FIG. 1] in which the reaction region is composed of a fluidization section I (fast bed) and of a section 2 formed by a riser. The feed gas 5 is introduced at the fluidized bed 1 and the oxidization of the propane takes place in the fluidized bed and in the riser 2.

A separation/stripping (stripper) unit 3, which can in particular be formed of a stripper and a series of cyclones, makes it possible to separate the reduced solid catalyst and the gaseous effluents resulting from the reaction region. The stripping gas 6 is an inert gas, preferably dry nitrogen or air, steam or a mixture of nitrogen or of air and of steam. The acrylic acid produced is recovered from the gaseous effluents leaving the unit 3.

The reduced solid is transported to the regeneration region 4, which consists of a fluidized bed section, where it is reoxidized in the presence of a mixture 7 composed of air, of oxygen-enriched air or of humid air. Preferably, the mixture is composed of air. The solid thus regenerated is subsequently recycled in the fluidization section 1.

It is advantageous to maintain the pressure of the reactor at from 1 to 5 bar and the temperature at between 250 and 450° C.

The reaction gas is introduced into the fluidized bed 1 with a total throughput which corresponds to the contact time of the gas respectively in the fluidized bed 1 and in the riser 2.

According to the invention, the proportions of the constituents of the starting gas mixture (5) can vary from 1/0-2/0-10 (in molar ratios), preferably propane/oxygen/inert gas(N₂) 1/0.05-2/1-10. (It being understood that these proportions do not take into account the recycling gases).

Under more particularly preferred conditions, they are from 1/0.1-1/1-5.

The active solid catalyst is also fed to the small fluidized bed 1.

On departing from the riser 2, the reaction gas and the reduced solid catalyst are separated in the stripping unit 3. The reduced solid catalyst is conveyed to the regenerator 4, where it is reoxidized under a mixture 7 preferably of air. It is subsequently recycled to the reaction region 1.

According to a preferred embodiment, the process is carried out in a circulating fluidized bed and in the absence of steam in the separating/stripping unit and/or in the regenerator.

The oxides of the various metals participating in the composition of the catalyst of formula (I) can be used as starting materials in the preparation of this catalyst but the starting materials are not limited to the oxides; other starting materials have been mentioned in international applications WO 04/024665 and WO 04/024666. The preparation of the catalysts and their regeneration have also been described in the above international applications or below in the examples.

The catalyst is regenerated according to the reaction (3):

SOLID_{reduced}+O₂→SOLID_{oxidized} (3)

by heating in the presence of air, of oxygen, of oxygen-enriched air or of a gas comprising oxygen, at a temperature of 250 to 500° C., for the time necessary for the reoxidation of the catalyst. Use is advantageously made of dry air (21% of O₂) or of humid air.

The proportions of the constituents of the regeneration gas mixture are generally as follows (in molar ratios):

oxygen/inert gas(N₂)/H₂O(steam)1/1-10/0-10.

According to another aspect of the invention, it is also possible to employ the process described in a circulating fluidized bed or in a fluidized bed, in the presence of a catalyst of general formula (I) as described above and of a cocatalyst as described in international application WO 03/45886, without introduction of steam into the initial gas mixture feeding the reaction.

### EXAMPLES

The following examples, given without implied limitation, show how the invention can be put into practice.

In the examples which follow, the conversions, selectivities and yields are defined as follows:

$$\begin{matrix}\begin{matrix}Conversion ( \% ) \\ of the propane\end{matrix} = \frac{\begin{matrix}Number of moles of propane \\ which have reacted\end{matrix}}{\begin{matrix}Number of moles of \\ propane introduced\end{matrix}} \times 100 \\ \begin{matrix}Selectivity ( \% ) \\ for acrylic acid\end{matrix} = \frac{\begin{matrix}Number of moles of acrylic \\ acid formed\end{matrix}}{\begin{matrix}Number of moles of propane \\ which have reacted\end{matrix}} \times 100 \\ \begin{matrix}Selectivity ( \% ) \\ for propionic acid\end{matrix} = \frac{\begin{matrix}Number of moles of propionic \\ acid formed\end{matrix}}{\begin{matrix}Number of moles of propane \\ which have reacted\end{matrix}} \times 100 \\ \begin{matrix}Selectivity ( \% ) \\ for acetone\end{matrix} = \frac{Number of moles of acetone formed}{\begin{matrix}Number of moles of propane \\ which have reacted\end{matrix}} \times 100\end{matrix}$$

The selectivities and yields relating to the other compounds are calculated similarly.

Conversion ratio (kg/kg)=weight of solid necessary to convert one kg of propane.

In the examples which follow, use is made of the technology described in international application WO 99/09809, which is incorporated here by way of reference and which will be referred to for all the operating details. In this technology, use is made of a circulating fluidized bed reactor [FIG. 1] in which the reaction region is composed of a fluidization section 1 (fast bed) and of a section 2 formed by a riser, the diameter/height ratio of which is in the proportions 15.6 mm/3 m. The feed gas 5 is introduced at the fluidized bed 1 and oxidation of the propane takes place in the fluidized bed and in the riser 2.

A separation/stripping (stripper) unit 3, which can in particular be formed of a stripper with a diameter of 100 mm and of a series of cyclones, makes it possible to separate the reduced solid catalyst and the gaseous effluents resulting from the reaction region. The stripping gas 6 is an inert gas, such as dry nitrogen, steam or a mixture of nitrogen and of steam. The acrylic acid produced is recovered from the gaseous effluents leaving the unit 3.

The reduced solid is transported to the regeneration region 4 or regenerator, which consists of a fluidized bed section with a diameter of 113 mm, where it is reoxidized in the presence of a mixture 7 composed of air, of oxygen-enriched air or of humid air. Preferably, the mixture 7 is composed of dry air. The solid thus regenerated is subsequently recycled in the fluidization section 1.

The pressure of the reactor is maintained at 2 psig (i.e., 1.09 bar absolute) and the temperature between 250 and 450° C. The balances are made after stabilizing for 30 min to 1 hour.

On departing from the riser 2, the reaction gas and the reduced solid catalyst are separated in the stripping unit 3. The gas phase is subsequently analyzed by gas chromatography, while the reduced solid catalyst is conveyed to the regenerator 4, where it is reoxidized under a mixture 7 of air (50% minimum) and optionally of steam, and with a total throughput of 700 NI/h. It is subsequently recycled to the reaction region 1.

The residence time of the solid in the unit 3 is between 1 and 6 minutes, preferably 4 minutes, and, in the unit 4, it is between 1 and 10 minutes, preferably 6 minutes.

### Examples 1 and 2

Examples 1 and 2 below are composed of several series of tests, the operating conditions and results of which are respectively summarized in tables 1 and 2. The catalyst employed is an antimony catalyst with a structure

Mo₁V_{0.3}Sb_{0.15}Nb_{0.1}Si_{0.93}Oₓ

The operating conditions given below are common to all the examples 1 to 7:

T_{reaction}=370° C.; (T_{regenerator}=370° C.)

Pressure=10⁵ Pa;

Feed throughput (5) of unit 1=600 NI/h; C₃H₈/O₂ (% volume/% volume) 20/18;

Total throughput (7) at the regeneration (unit 4)=700 Sl/h

Stripper total throughput (6) (unit 3)=740 NI/h

Throughput for circulation of the solid=37 kg/h

Conversion ratio of the order of 700 kg of catalyst/kg of propane converted. This parameter reflects the amount of catalyst necessary to convert 1 kg of propane.

The feed gas for section 1 is composed of a C₃H₈/O₂/N₂/(H₂O—comparative tests) mixture, the proportions of which are shown in the various tables, the nitrogen acting as remainder to 100%.

In each series, a reference test was carried out in order to make sure that the catalyst was not deactivated.

### Example 1

TABLE 1

| Example No. | | | |
|---|---|---|---|
| | Comparative | | |
| 1 | 1a | 1b | 1c |

Operating conditions

| | | | | |
|---|---|---|---|---|
| % H₂O in (5) | 0 | 10 | 25 | 50 |
| % H₂O in (6) | 50 | 50 | 50 | 50 |
| % H₂O in (7) | 0 | 0 | 0 | 0 |

Results

| | | | | |
|---|---|---|---|---|
| C₃H₈ conversion (%) | 20.8 | 22.1 | 23.9 | 22.9 |
| Select. (acrylic acid + propylene) | 55.0 | 54.2 | 52.3 | 53.5 |
| (%) | | | | |
| Select. acrylic acid (%) | 42.6 | 39.4 | 40.5 | 41.9 |
| Select. CO₂ + CO (%) | 34.1 | 34.9 | 35.6 | 31.2 |
| Select. propionic acid (%) | 0.10 | 0.10 | 0.13 | 0.55 |
| Select. acetic acid (%) | 10.3 | 10.1 | 11.3 | 13.6 |
| Select. acetone (%) | 0.47 | 0.53 | 0.56 | 1.07 |
| Conversion ratio (kg/kg) | 750 | 710 | 654 | 683 |

This example shows, by means of the comparative tests, that the presence of water in (5), the gas stream feeding the reaction, promotes the formation of hydration products (acetone and propionic acid).

### Example 2

TABLE 2

| Example No. | | | |
|---|---|---|---|
| | Comparative | | |
| 2 | 2a | 2b | 2c |

Operating conditions

| | | | | |
|---|---|---|---|---|
| % H₂O in (5) | 0 | 15 | 25 | 50 |
| % H₂O in (6) | 0 | 0 | 0 | 0 |
| % H₂O in (7) | 0 | 0 | 0 | 0 |

Results

| | | | | |
|---|---|---|---|---|
| C₃H₈ conversion (%) | 18.1 | 19.8 | 21.7 | 22.3 |
| Select. (acrylic acid + propylene) | 51.8 | 48.7 | 49.7 | 51.7 |
| (%) | | | | |
| Select. acrylic acid (%) | 37.5 | 34.7 | 37.3 | 39.5 |
| Select. CO₂ + CO (%) | 39.7 | 42.6 | 40.1 | 33.1 |
| Select. Propionic acid (%) | 0.06 | 0.07 | 0.09 | 0.42 |
| Select. acetic acid (%) | 8.0 | 8.2 | 9.6 | 13.7 |
| Select. acetone (%) | 0.43 | 0.49 | 0.49 | 1.07 |
| Conversion ratio (kg/kg) | 865 | 794 | 722 | 773 |

This example shows, by means of the comparative tests, that the presence of water in (5), the gas stream feeding the reaction, promotes the formation of hydration products (acetone and propionic acid).

### Examples 3 and 4

Results obtained after a test lasting 24 hours carried out in the complete absence of feeding with water.

TABLE 3/4

| Example No. | | |
|---|---|---|
| | 4 | 4 |
| 3 | t = 0 | t = 24 h |

Operating conditions

| | | | |
|---|---|---|---|
| % H₂O in (5) | 0 | 0 | 0 |
| % H₂O in (6) | 50 | 0 | 0 |
| % H₂O in (7) | 0 | 0 | 0 |

Results

| | | | |
|---|---|---|---|
| C₃H₈ conversion (%) | 20.8 | 19.2 | 18.9 |
| Select. (acrylic acid + propylene) (%) | 55.0 | 49.9 | 50.6 |
| Select. acrylic acid (%) | 42.6 | 35.7 | 35.7 |
| Select. CO₂ + CO (%) | 34.1 | 41.5 | 41.3 |
| Select. propionic acid (%) | 0.10 | 0.06 | 0.06 |
| Select. acetic acid (%) | 10.3 | 8.1 | 7.6 |
| Select. acetone (%) | 0.47 | 0.44 | 0.43 |
| Conversion ratio (kg/kg) | 752 | 818 | 826 |

It is found that the performances do not change over time. The activity of the catalyst does not deteriorate after operating without water. The propionic acid content is minimal.

### Example 5

Catalyst A with antimony. Tests carried out in the absence of oxygen and with vol % of propane in the feed (5) of the fluidized bed (1):

TABLE 5

Example No.

| | |
|---|---|
| | Comparative |
| 35 | 5a |

Operating conditions

| | | |
|---|---|---|
| % H₂O in (5) | 0 | 15 |
| % H₂O in (6) | 40 | 40 |
| % H₂O in (7) | 0 | 0 |

Results

| | | |
|---|---|---|
| C₃H₈ conversion (%) | 23.8 | 25.8 |
| Select. (acrylic acid + propylene) (%) | 45.7 | 50.4 |
| Select. acrylic acid (%) | 34.4 | 39.3 |
| Select. CO₂ + CO (%) | 44.0 | 34.2 |
| Select. propionic acid (%) | 0.04 | 0.11 |
| Select. acetic acid (%) | 8.7 | 13.8 |
| Select. acetone (%) | 0.4 | 0.7 |
| Conversion ratio (kg/kg) | 2600 | 2400 |

The content of propionic acid and of acetone is minimal. A particularly high conversion ratio is observed

### Examples 6 and 7

Catalysts with tellurium: Mo₁V_{0.33}Te_{0.22}Nb_{0.11}Si_{1.11}Oₓ. These tests comprise tests carried out with a propane/oxygen ratio (vol %)=20/18 in the feed (5) of the fluidized bed (1) and with a water feed of 50% in the unit 3 and in the absence of water, also in the regeneration (unit 4),

TABLE 6/7

| Example No. | | | |
|---|---|---|---|
| Comparative | Comparative | | |
| 6/7a | 6/7b | 6 | 7 |

Operating conditions

| | | | | |
|---|---|---|---|---|
| % H₂O in (5) | 10 | 10 | 0 | 0 |
| % H₂O in (6) | 50 | 50 | 50 | 50 |
| % H₂O in (7) | 0 | 0 | 0 | 0 |

Results

| | | | | |
|---|---|---|---|---|
| C₃H₈ conversion (%) | 28.6 | 29.1 | 27.8 | 27.6 |
| Select. (acrylic acid + | 54.4 | 55.8 | 54.2 | 54.3 |
| propylene) (%) | | | | |
| Select. acrylic acid (%) | 44.8 | 46.9 | 44.2 | 44.4 |
| Select. CO₂ + CO (%) | 37.0 | 36.3 | 39 | 39.9 |
| Select. propionic acid | 0.14 | 0.13 | 0.09 | 0.09 |
| (%) | | | | |
| Select. acetic acid (%) | 7.5 | 7.6 | 5.5 | 5.4 |
| Select. acetone (%) | 0.40 | 0.40 | 0.32 | 0.32 |
| Conversion ratio (kg/kg) | 550 | 540 | 560 | 570 |

A reduced formation of hydration products is found in tests 6 and 7 for a comparable selectivity for acrylic acid.

### Preparation of the Catalysts

1. Preparation of Catalyst A with a structure: Mo₁V_{0.3}Sb_{0.15}Nb_{0.1}Si_{0.9}Oₓ

### Preparation of the Solution B

The following are introduced into a Rayneri Trimix mixer:

295 g of niobic acid (HY-340 CBMM, 81.5% Nb₂O₅)

660 g of oxalic acid dihydrate (Prolabo)

5 liters of water

It takes two hours for the niobic acid (Nb₂O₅ hydrate) to dissolve at 65° C. The molar ratio of the oxalic acid to the niobium is 3. The solution is collected and stored and will be used in its entirety.

### Preparation of the Solution A

3090 g of ammonium heptamolybdate (Starck)

615 g of ammonium metavanadate (GfE)

385 g of antimony oxide (Sb₂O₃, Campine)

9750 g of demineralized water

The solution is heated with stirring at 99° C. for three hours after stabilization of the temperature. An opaque mixture with a dark blue color is obtained.

348 g of 30% aqueous hydrogen peroxide solution are added, so as to obtain a clear solution with an orange color.

### Addition of Colloidal Silica

2455 g of Ludox colloidal silica (Grace, AS-40) comprising 40% by weight of SiO₂ are added to the solution A without modifying the appearance of the mixture, which remains clear.

### Formation of the Suspension

The solution B of oxalic acid and of niobic acid is poured into the solution A/colloidal silica mixture. The mixture turns cloudy with the formation of a precipitate in suspension and the color becomes orangey yellow. Precursor fines (1370 g) originating from the preceding atomization operation are added to the solution at this stage. After stirring for an additional half hour, heating is halted. The suspension is then recovered and micronized. The d50 (mean diameter of the particles in suspension, measured by laser particle sizing on a Horiba LA300) changes from 18 μm to 0.2 μm with the micronization.

### Micronization

The micronization is carried out on a Labstar apparatus from Netzsch under the following operating conditions:

Mill speed: 3500 rev/min

Feed pump indicator: 75 rev/min

The outlet temperature of the product reaches 55° C.

The micronized suspension is immediately atomized (dry matter content of the mixture, measured with an infrared dryer, at 33% by weight).

### Atomization

The atomization operation is carried out immediately after the micronization. A Niro Minor Mobile High-Tech atomizer is used. The drying chamber has a Jacket heightened by 2 m through which the steam passes. The drying gas is nitrogen. The spray nozzle is based on the principle of the generation of droplets by vibrations resulting from an ultrasound generator (Sodeva, ultrasound frequency: 20 kHz). The feed tank is kept stirred and the suspension is preheated to 60° C. using a thermostatically-controlled bath. The operating conditions are:

T° C. inlet: 210° C.

T° C. outlet: 105° C.

Feed throughput: 5.5 kg/h on average

Nitrogen throughput: 80 m³/h

The size distribution of the particles is analyzed by laser particle sizing after drying overnight in an oven at 80° C. The solid is subsequently sieved so as to remove as much as possible of particles with a diameter of less than 50 μm and also the particles of greater than 160 μm.

### Heat Treatments

The heat treatment is carried out using a rotating oven (200 mm in diameter, 270 mm cylinder length, working volume of 2.5 liters). One of the ends is closed. The gas is introduced using a pipe as far as the inside of the cylinder.

3319 g of solid are first treated at 310° C. [300-310] under 900 l/h [100-1200] of air for 4 hours and then at 600° C. under nitrogen (200 l/h) for two hours. The temperature gradient is 4.5° C./min in the solid, on average. An oximeter connected to the nitrogen supply system measures the oxygen content of the gas: typically between 1 and 2 ppm. The rotational speed of the oven is 15 rev/min.

2630 g are recovered. A final sieving is carried out in order to retain only the fraction from 50 to 160 μm: 2261 g.

The catalyst A is composed of 6 batches resulting from analogous preparations.

### Properties of the Catalyst A

Particle sizing of the catalyst A—measured by laser particle sizing on a Horiba LA300

D50=68 μm (mean diameter of the particles)

>160 μm=2% by weight (particles of more than 160 microns)

<50 μm=10% by weight (particles of less than 50 microns)

bulk density (measured by the method described in standard ISO 3923/1) 1.45 g/cm³

Spheres: ratio of the Feret's diameters: 1.1

2. Preparation of Catalyst 13 with the Formulation

Mo₁V_{0.33}Te_{0.22}Nb_{0.11}Si_{1.11}Oₓ

### Preparation of the Solution B

The following are introduced into a Rayneri Trimix mixer:

295 g of niobic acid (HY-340 CBMM, 81.5% Nb₂O₅)

660 g of oxalic acid dihydrate (Prolabo)

5 liters of water

It takes two hours for the niobic acid (Nb₂O₅ hydrate) to dissolve at 65° C. The molar ratio of the oxalic acid to the niobium is 3. The solution is collected and stored and will be used in its entirety.

### Preparation of the Solution A

2819 g of ammonium heptamolybdate (Starck)

616 g of ammonium metavanadate (GE)

802 g of telluric acid (H₆TeO₆, Fluka)

4061 g of demineralized water [variation in the amount of water according to the preparations by a factor of 1 to 3; here, 4 l represents the lowest amount]

The solution is heated for one hour at 90-95° C. with stirring until dissolution is complete and a clear orange-red solution is obtained.

### Addition of Colloidal Silica

2655 g of Ludox colloidal silica (Grace, AS-40) comprising 40% by weight of SiO₂ are added to the solution A without modifying the appearance of the mixture, which remains clear.

### Formulation of the Suspension

The solution B of oxalic acid and of niobic acid is poured into the solution A/colloidal silica mixture. The mixture turns cloudy with the formation of a precipitate in suspension and the color becomes orangey yellow. After stirring for an additional half hour, heating is halted. The suspension is then recovered and immediately atomized (dry matter content of the mixture, measured using an infrared dryer, at 36% by weight).

### Atomization

The atomization operation is carried out immediately after the preparation of the suspension. The Niro Minor Mobile High-Tech atomizer, modified internally, is preferably used. The drying gas is nitrogen. The drying chamber, heightened by 2 m, has a jacket through which steam passes. The spray nozzle is based on the principle of the generation of droplets by vibrations resulting from an ultrasound generator (Sodeva, ultrasound frequency: 20 kHz). The feed tank is kept stirred and the suspension is preheated to 60° C. using a thermostatically-controlled bath. The conventional operating conditions are.

T° C. inlet: 209-210° C.

T° C. outlet: 105-110° C.

Feed throughput: 5 kg/h on average

Nitrogen throughput: 80 m³/h

The evaporative capacity of the atomizer is 3 kg/h of water.

The solid recovered is subsequently further dried overnight in a ventilated oven at 80° C. The solid is subsequently sieved, so as to remove as much as possible of particles with a diameter of less than 50 μm and also the particles of greater than 160 μm.

### Heat Treatments

The heat treatment is carried out using a rotating oven (200 mm in diameter, 270 mm cylinder length, working volume of 2.5 liters). One of the ends is closed. The gas is introduced using a pipe as far as the inside of the cylinder. Various batches analogously treated were combined (air throughput 150 l/h (100 and 400 l/h), precalcination temperature 300° C., nitrogen throughput 150 or 200 I/h, calcination temperature 600° C., temperature gradient approximately 3.5 to 4.5° C./min).

2913 g of calcined solid were discharged after treating 3.805 kg of solid. A final sieving is necessary in order to retain only the 50-160 μm fraction.

This preparation was repeated several times in order to obtain 10 kg of catalyst, which were homogenized before use.

### Properties of the Catalyst B

Final particle size distribution (but the pilot-plant team receives before charging):

D50 (mean diameter of the particles, measured using a Horiba LA300)=71 μm

<50 μm (particles of less than 50 microns−fines)=15% by weight

>160 μm (particles of more than 160 microns)=1% by weight

Bulk density (measured by the method described in standard ISO 3923/1): 1.40 g/cm³.

Spheres: ratio of the Feret's diameters=1.3.

1. A process for the preparation of acrylic acid comprising a reaction of selectively oxidizing propane in an initial gas mixture, in a circulating fluidized bed or in a non-circulating fluidized bed, in the presence of a catalyst with the structure:
Mo₁VₐX_{b}Z_{c}Si_{d}Oₓ (I)
in which X is tellurium or antimony and Z is niobium or tantalum, and in which:
a is between 0.006 and 1, limits included;
b is between 0.006 and 1, limits included;
c is between 0.006 and 1, limits included;
d is between 0 and 3.5, limits included; and
x is the amount of oxygen bonded to the other elements and depends on their oxidation states,
under conditions for the partial conversion of the propane and without introduction of steam into the initial gas mixture feeding the reaction.

2. The process as claimed in claim 1, characterized in that the initial gas mixture is composed of a propane/oxygen/inert gas mixture.

3. The process as claimed in claim 1, characterized in that the gas mixture feeding the reaction also comprises recycling gases.

4. The process as claimed in claim 1, wherein the catalyst corresponds to the formula (I) defined in claim 1, in which X represents antimony.

5. The process as claimed in claim 1, wherein the catalyst corresponds to the formula (I) defined in claim 1, in which X represents tellurium.

6. The process as claimed in claim 1, wherein the catalyst corresponds to the formula (I) defined in claim 1, in which Z represents niobium.

7. The process as claimed in claim 1, wherein the catalyst corresponds to the formula (I) defined in claim 1, in which Z represents tantalum.

8. The process as claimed in claim 1, wherein the selective oxidation of the propane is carried out in a circulating fluidized bed.

9. The process as claimed in claim 1, wherein the process is carried out in a circulating fluidized bed and in the absence of steam in the separation/stripping unit and/or in the regenerator. ator.

10. The process as claimed in claim 1, wherein the selective oxidation of propane is carried out in the presence of a cocatalyst.
